(19) United States
(12) Patent Application Publication
(10) Pub. No.: US 2025/0057795 A1
(43) Pub. Date: Feb. 20, 2025

(54) LIQUID COMPOSITIONS, EYE DROPS, AND METHODS FOR TREATMENT OF EYE DISEASES USING Levodopa FORMULATIONS

(71) Applicant: JOHN A. MARTENS, TUCSON, AZ (US)

(72) Inventor: JOHN A. MARTENS, TUCSON, AZ (US)

(21) Appl. No.: 18/802,213

(22) Filed: Aug. 13, 2024

Related U.S. Application Data
(60) Provisional application No. 63/532,641, filed on Aug. 14, 2023.

(51) Int. Cl. A61K 31/195; A61K 9/08; A61K 9/107; A61K 31/355; A61K 31/375; A61K 31/4045; A61K 31/714; A61K 31/728; A61K 38/06

(52) U.S. Cl. CPC A61K 31/195; A61K 9/08; A61K 9/107; A61K 31/355; A61K 31/375; A61K 31/4045; A61K 31/714; A61K 31/728; A61K 38/063

(57) ABSTRACT

Therapeutic eye drops target the treatment/prevention of age-related macular degeneration, diabetic retinopathy and myopia with formulations designed for topical application. The formulations employ a combination of anti-oxidants that act to stabilize the formulations and prevent oxidation of other key components. Long term stability for patients and in-clinical use are critical predications for routine use in an ambulatory setting over a long period of time.

### BACKGROUND OF THE INVENTION

Vision loss due to eye diseases is a global health issue that affects over a billion people worldwide who have a near or distance vision impairment that could have been prevented or has yet to be addressed in the absence of timely detection. Vision impairment affects people of all ages, with the majority being over 50. Young children with early onset severe impairment can experience lower levels of education achievement, and for adults it often affects quality of life through lower productivity, decreased workforce participation, and high rates of depression.

Vision impairment and blindness impact the life of people everywhere in low- and middle-income settings, the burden of vision impairment can even be greater due to fewer opportunities to access the most essential eye care services.

Cataracts and uncorrected refractive errors are estimated to be the leading causes of vision impairment, however, other causes for vision impairment cannot be ignored. Age related macular degeneration, glaucoma, long standing systemic conditions like diabetes causing diabetic retinopathy, infectious diseases of the eye and trauma to the eye are all equally important causes for vision impairment that need addressing. The (1) billion cited above includes those with moderate or severe distance impairment or blindness dye to unaddressed refractive error (88.4 million), cataract (94 million), age-related macular degeneration (20+million), glaucoma (7.7 million), diabetic retinopathy (3.9 million) as well as near vision impairment caused by unaddressed presbyopia (826 million).

Blinding eye diseases, including age-related macular degeneration (AMD), affect more than 11 million Americans. Age-related macular degeneration (AMD), cataracts, diabetic retinopathy. glaucoma, and myopia collectively impact ˜10% of the world's population. Effective treatments have been elusive and out of reach for many of the affected due to cost, lack of local specialized ophthalmology care, limitations in medical insurance coverage, a lack of focus on prevention and early intervention, and profits over the eye health of populations in general. The situation varies considerably depending on where the need exists. Hence, there is an acute need for more effective, readily available, cost effective treatments that have proven to be elusive.

Brief comments about each of the above conditions will increase appreciation for their impact and the status of options for treatment.

### SUMMARY OF THE INVENTION

The present invention relates to a composition for direct, topical treatment of eye disease conditions resulting from risk factors that include smoking, aging, and light (“blue”) eye pigmentation. Stable compositions of these formulations included within the scope of the invention.

An aqueous liquid composition for use in treating genetically resultant conditions in the eye comprising (L-DOPA, aka Levodopa) and an antioxidant combination of glutathione (γ-Glutamylcysteinylglycine) and ascorbic acid with Vitamin B12 as a co-factor. Additional antioxidants, including Vitamin E, and melatonin can also be included. As emulsions, the formulations are best prepared by a two step process, starting with Part A containing aqueous soluble components, namely Levodopa, carbidopa, Vitamin C, and glutathione along with melatonin (as needed). Melatonin is insoluble in water, but its incorporation into the solution will be exemplified in Part A of the detailed example that follows. Part A is prepared by adding the components to distilled, sterile water and mixing until dissolution is complete (typically a few minutes). Next Part B is prepared in a separate vessel by adding the emulsifier (Tween 80 or a Pluronic polyol). Adding Part A to Part B with mixing generates the emulsion, followed by raising the pH to the range of 6.8 to 7.5 that can be easily accomplished with NaHCO₃ or other buffering agents used routinely in therapeutic eye drops.

The inventive eye drop formulations are stable as eye drops and are also designed to restore redox balance to eye tissues that are deficient in essential antioxidant function tied to the eye diseases targeted by this invention.

An aqueous liquid composition used to treat genetically resultant conditions in the eyes of mammals comprises LevoDOPA and its neurotransmitter functional derivatives, including prodrugs (2-O-methyl DOPA and 2-O-ethylDOPA) and an antioxidant combination of glutathione and ascorbic acid (inclusive of ascorbates, unless otherwise specified). The composition is preferred wherein the dopaminergic agent is selected from the group consisting of levodopa, carbidopa, levodopa metabolite, 3-O-methyldopa and 3-O-ethyldopa.

### BRIEF DESCRIPTION OF THE FIGURES TABLES

FIG. 1 is an image of Redox Balancing: Link between Vitamin C, Glutathione, Vitamin B12.

FIG. 2 is an image of Vitamin C/Glutathione/Vitamin B12 Redox Biochemical Cycle.

FIG. 3 shows Tear Film Structure.

FIG. 3A shows three tables of therapeutic formulations 3A, 3B and 3C,

FIG. 4A shows a second set of Therapeutic Eye Drop Formulations.

FIG. 5A shows a series of Therapeutic Eye Drop formulations 5G, 5H and 5I within the scope of the invention.

FIG. 6—Table 6-G,H,I shows Therapeutic Formulations within the scope of the invention.

### DETAILED DESCRIPTION OF THE INVENTION

The present invention relates to the treatment of the genetic conditions or diseases of the eyes of mammals by applying medicinal liquid drops directly onto the surface of the eye. The treatment is believed useful in at lest mediating numerous genetic conditions. The drops are applied as a liquid and may be solutions, emulsions or suspensions of the necessary ingredients. Solutions and emulsions are preferred to avoid storage problems with the medicine (such as deposition of solid within the container or dropper) when solids are used.

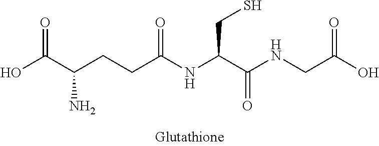 [SMILES: N[C@@H](CCC(=O)N[C@@H](CS)C(=O)NCC(=O)O)C(=O)O]

We believe an eye drop would be an ideal delivery mechanism. An eye drop delivers the drug directly to the organ of interest while reducing the risk of systemic toxicity or side effects. Also, and critically so, an eye drop may be more widely accepted by patients who have difficulty swallowing pills and more easily associated as a benefit to the eye. The inventors also believe that a new combination of drugs with L-DOPA may prevent or significantly delay the onset of wet AMD or reduce and extend the need for treatments for patients already diagnosed with nAMD. We have developed a unique liquid emulsion of ingredients that prevent oxidation of L-DOPA, enhance the bioavailability of L-DOPA (carbidopa decreases dopa decarboxylase conversion of L-DOPA to dopamine) has appropriate tonicity and pH. We have also discovered that we can add other components such as the active ingredients of the clinically proven AREDS and AREDS 2 that can complement the action of L-DOPA and enhance the patient compliance by reducing the number of individual medications. We have determined that we can add antioxidant agents such as CoQ10 or ubiquinol (reduced form of CoQ10) that may also enhance the protection against AMD or other oxidative pathologies. Finally our emulsions with L-DOPA and antioxidants may be effective at treating or preventing glaucoma, preventing cataracts, preventing or treating uveitis, pterygium pinguecula, or delivering antibiotics, antifungals, anti-inflammatory drugs, steroids, anti dry eye agents, or drugs that enhance meibomian function to the eye and other diseases that may benefit from these agents and their delivery via the nano-emulsions described and defined herein by this invention. Thus our new emulsion systems is and formulations are not limited to use in preventing or treating neovascular AMD.

The evidence that these drugs will work begins with the discovery that L-DOPA is the ligand for GPR143, the ocular albinism gene. L-DOPA stimulation of retinal pigment epithelial cells internalized GPR143 and stimulated the release of PEDF (pigment epithelium derived factor), a potent neurotrophic factor, and down regulated the release of exosomes and VEGF, which, in excess, stimulates neovascular vessel growth in nAMD. It was observed that dopamine-bound GPR143 at the same site with a near-equal affinity to L-DOPA stimulated the release of VEGF and exosomes—the significance of which will be discussed later in the proposal. Based on the findings with GPR143, Dr. Mckay and John Martens, Snyder Biomedical co-founder, hypothesized in their PCT Patent Application WO2009129947A2 that DOPA may be protective for both dry and wet AMD because of the neurotrophic factor PEDF as well as down-Regulating VEGF.

Since L-DOPA is a commonly used pharmaceutical to treat movement disorders like Parkinson's disease, others performed a retrospective analysis of three different cohorts to evaluate if individuals taking L DOPA for Parkinson's or other motor problems were protected from AMD, likely from L-DOPA activation of GPR143 in the retinal pigment epithelium. They examined clinical data encompassing more than one million individuals. People with a history of Levodopa prescriptions were significantly less likely to develop any type of AMD. Further, if they did develop the disease, the age of onset was significantly delayed by eight years. If they looked specifically at nAMD, there was a five-year delay in AMD (OR 0.65; 95% CI, 0.65-0.69; P<. 001). This led to the conclusion that Levodopa supplementation protects against developing AMD and nAMD, and that a prospective study was warranted. In a subsequent open-label, “proof-of-concept” study that Snyder Biomedical (SBC) conducted, patients with newly diagnosed nAMD who were naive to anti-VEGF injections (Cohort-1), the effects of Carbidopa/Levodopa on vision and anatomic outcomes were evaluated for four weeks.10 Patients were then followed five months further with ascending Levodopa doses. Patients previously treated with anti-VEGF injection therapy (Cohort-2) were also treated with ascending Levodopa doses and evaluated for six months. SBC found that LDOPA/Carbidopa was safe, well-tolerated, and delayed anti-VEGF injection therapy while improving visual outcomes.

Carbicopa (C₁₀H₁₄N₂O₄) increases the plasma half-life of levodopa from 50 minutes to 1½ hours. Carbidopa is an aromatic L-amino acid dicarboxylase inhibitor (DOPA decarboxylase or DDC) and has been used for over 60 years in the treatment of Parkinsonism in combination with L-Dopa. The original product developed for this use is Sinemet™ product.

In the first month, retinal fluid decreased by 29% (P=0.02, n=12),) 10 without anti-VEGF injections. Through six months, the decrease in retinal fluid in Cohort 1 was sustained, with a mean frequency of 0.38 injections/month. At month 6, mean visual acuity improved by 4.7 letters in Cohort-1 (P=0.004, n=15) and by 4.8 letters in Cohort-2 (P=0.02,−=11). Additionally, there was a 52% reduction in the need for anti-VEGF injections in Cohort-2 (P=0.002). Our findings suggested efficacy and support for the pharmacological targeting of GPR143 in placebo-controlled and randomized clinical trials.

We have designed such a clinical trial study but plan to use Etilevodopa (aka 2-O-ethylDOPA) as a pill and formulated as an eye drop. Etilevodopa has demonstrated increased ocular penetration due to increased solubility over Levodopa/Carbidopa is useful because it prevents the conversion of L-DOPA to dopamine in the peripheral circulation and tear film, enhancing the amount of L-DOPA available to bind GPR 143. Melatonin, on the other hand, is a circadian signal that shuts down dopamine released during the day and resets the retinal circadian clock. As mentioned earlier, dopamine also binds GPR143 to enhance VEGF secretion. Therefore, melatonin via dopamine downregulation should synergistically reduce VEGF in a natural manner when dosed in the evening. This has been validated in an RPE culture and continuous perfusion system, alternating L-DOPA/melatonin supplementation with dopamine, and found the RPE cells could be induced to release VEGF in a diurnal manner. We previously submitted a U.S. patent application PCT/US2020/015860, now US20220023245, filed Jan. 30, 2020) for the “melatonin effect” on VEGF and a L-DOPA/carbidopa and melatonin formulation should be a unique new drug for AMD with new patent protection. This application is incorporated in its entirety by reference herein. In addition, melatonin also serves as an important antioxidant and may stabilize liquid formulations of L-DOPA including solutions and emulsions containing it.

### Additional Utility for an L-DOPA/Carbidopa Eye Drop

The inventors also believe the eye drop formulations will be useful to treat at least two additional eye diseases, including diabetic retinopathy, a complication of diabetes and myopia. While myopia does not cause blindness, it strongly affects vision and becomes burdensome over time, particularly for young children when it is typically diagnosed around age 5 or higher.

### Diabetic Retinopathy

Diabetes is a metabolic disease caused by dysfunction in the conversion of glucose to ATP responsible for energy generation at the cellular level in all mammals. The conversion is catalyzed by insulin, a hormone produced endogenously by the pancreas. In non-diabetic people, the conversion happens in direct response to blood glucose concentration tied to the consumption of carbohydrates and sugars. Hyperglycemia is a direct result of inadequate production of insulin to meet the demands for converting amounts of sugars and carbs. Persistent high blood sugar, if untreated, will lead to diabetes and serious health consequences as a result. Individuals that develop diabetes later in life are diagnosed with Type 2 diabetes, while the emergence of diabetes at a young age is usually diagnosed as Type I diabetes.

Those with Type I diabetes require daily injections of insulin to support the metabolism of the body. For Type II diabetes, depending on its severity and longevity, those diagnosed are prescribed at least one medication to promote insulin by the pancreas. The most common medication is metformin, an aldose reductase inhibitor, that regulates the AR enzyme that converts glucose to sorbitol. in cells. Metformin helps control high blood sugar reducing kidney damage, blindness, nerve issues, loss of limbs, etc.

Diabetic retinopathy (DR), the most common microvascular complication that occurs in diabetes mellitus (DM), is the leading cause of vision loss in working-age adults. The prevalence of diabetic retinopathy is approximately 30% of the diabetic population and untreated DR can eventually cause blindness. For decades, diabetic retinopathy was considered a microvascular complication and clinically staged by its vascular manifestations. In recent years, emerging evidence has shown that diabetic retinopathy causes early neuronal dysfunction and neurodegeneration that may precede vascular pathology and affect retinal neurons as well as glial cells. This knowledge leads to new therapeutic strategies aiming to prevent dysfunction of retinal neurons at the early stage of DR. Early detection and timely treatment to protect retinal neurons are critical to preventing visual loss in DR. This review provides an overview of DR and the structural and functional changes associated with DR, and discusses neuronal degeneration during diabetic retinopathy, the mechanisms underlying retinal neurodegeneration and microvascular complications, and perspectives on current and future clinical therapies.

DR is a leading cause of blindness in the world ranging from working-age adults to the elderly population (20-74 years old). It is estimated that the DR population worldwide will increase from 463 million in 2019 to 578 million in 2030 and to approximately 700 million by 2045. A study showed that more than 30 million people (˜9.4% of the US population) have diabetes, and approximately one-third of them are diagnosed with diabetic retinopathy. DR patients can suffer severe vision loss if left untreated. DR is also associated with the risks of systemic vascular complications of diabetes, including stroke, cardiovascular events and heart failure. Diabetes affects all cells in the retina, though most studies have focused mainly on retinal microvascular pathology. Based on the presence of neovascularization, DR is classified into two stages, non-proliferative (NPDR) and proliferative diabetic retinopathy (PDR). NPDR is an early stage of DR. Early morphological signs of NPDR include basal membrane thickening, tight junction impairment, and blood-retina barrier (BRB) breakdown. Vascular lesions accumulate to induce ischemic conditions in some areas of the retina; as ischemia develops, proangiogenic factors such as vascular endothelial growth factor (VEGF) release, inducing the formation of neovascularization, a hallmark of proliferative DR. Drug treatment has been an emerging therapy to treat DR such as anti-VEGF or steroid drugs, which also target end-stages of the disease after damage has already occurred. Leading clinical anti-VEGF drugs including ranibizumab, bevacizumab, and aflibercept, have been widely utilized to treat DR patients. Early anti-VEGF injection, before complications of DR have developed, can reduce further progression into severe stages.

As the Mckay team demonstrated in-vitro (McKay, B. S. et al., L-Dopa is an Endogenous Ligand for OA1, PlosBio 2008 Sep. 30, 6 (9): e236.), L-DOPA binds as a ligand with the GPR143 receptor in RPE cells and shuts down VEGF expression as a consequence of ligation. Delivery of L-DOPA via a topical eye drop is therefore a potential therapeutic strategy for treating diabetic retinopathy. Delivery of L-DOPA via a topical eye drop is therefore a potential therapeutic strategy for treating diabetic retinopathy. The penetration of L-DOPA (or an L-DOPA prodrug including Methyl DOPA or Etilevodopa) delivered to the scleral surface of the eye with transportation through the vitreous humor and to the retina is important for this therapeutic approach to function. We believe it is worth the effort, hence our belief this strategy can be effective in regulating DR.

### Myopia

Myopia is a visual disorder, often referred to as short-sightedness, caused by excessive elongation of the axial length of the eye during development. Myopia is the leading cause of poor vision and the most common eye disorder worldwide. Estimates are that it may impact ⅓ of the population by the end of the decade. Prevalence is highest in urban East Asia, where in many parts, as many as 80% of school attendants are myopic. The prevalence is strongly associated with the amount of time spent outside in natural bright light. Spending time outdoors is a potent anti-dote against developing myopia among children.

Animal studies show that it is related to light induced increases of dopamine levels within the eye. This suggests that the pre-cursor of dopamine, often referred to as a dopamine prodrug, L-DOPA is involved.

Public perception of myopia (nearsightedness) as a visual inconvenience masks the severity of its sight-threatening consequences. Myopia is a significant risk factor for posterior pole conditions such as maculopathy, choroidal neovascularization and glaucoma, all of which have a vascular component. These associations strongly suggest that myopic eyes might experience vascular alterations prior to the development of complications. Myopic eyes are out of focus because they are larger in size, which in turn affects their overall structure and function, including those of the vascular beds. Vascular manifestations of myopia are displayed.

For example, a manifestation of advanced myopia is choroidal neovascularization (CNV) that we have already established is a byproduct of overexpression of VEGF-vascular endothelial growth factor. CNV is characterized by an atypical choroidal vasculature growth into the retinal pigment epithelium potentially leading to fluid and blood accumulation in the macula. Eyes with lower foveolar choroidal blood volume and flow have been identified to be at a higher risk of developing CNV. This reduced choroidal blood supply appears greater than any changes observed in eyes without CNV, suggesting that alterations in the foveal choroidal circulation might precede be part of CNV etiology. In addition, the choroidal thinning and capillary density reduction observed in degenerative pathological myopia is believed to trigger RPE and glial cells hypoxia, resulting in an upregulation of VEGF expression.

Choroidal neovascularization (CNV) is characterized by an atypical choroidal vasculature growth into the retinal pigment epithelium potentially leading to fluid and blood accumulation in the macula. Eyes with lower foveolar choroidal blood volume and flow have been identified to be at a higher risk of developing CNV. This reduced choroidal blood supply appears greater than any changes observed in eyes without CNV, suggesting that alterations in the foveal choroidal circulation might precede be part of CNV etiology. In addition, the choroidal thinning and capillary density reduction observed in degenerative pathological myopia is believed to trigger RPE and glial cells hypoxia, resulting in an upregulation of VEGF expression.

It is the belief that the interaction between excessive myopic eye growth and vascular alterations are tipping-points for the development of sight-threatening changes over time.

The eye drops of the invention may benefit from the prodrug Etilevodopa over Levodopa in this application due to increased penetration in the eye following topical application at a high % solids. This will be verified as part of ongoing efforts. Furthermore, the present novel stabilization system to prevent oxidation of the DOPA components (Levodopa, Etilevodopa, Methyldopa and Carbidopa) are all substrates that require anti-oxidant protection for use in eye drop formulations targeting therapeutic use in a clinical or home use (by patients) setting.

### The Self-Emulsification Process and Formulations

Oil-in-water emulsions are widely used in ophthalmic products. Typical preparations of oil-in-water emulsions involve dissolving water-soluble components in an aqueous phase and dissolving oil-soluble components in an oil phase. The oil phase is then mechanically dispersed in a vigorous process into the aqueous phase, for example, by mixing at several thousand revolutions per minute (r.p.m.) for minutes to several hours. This process is called emulsification. Emulsification is an energy-consuming process, in which thermal and kinetic energies are invested in order to disperse the oil phase into numerous droplets of very small size but having a large total surface area.

Ocular comfort is of critical importance for commercial success in ophthalmic products. Emulsions containing a high concentration of oil (i.e., generally more than 6% v/v oil) are uncomfortable for ophthalmic uses. Therefore, the ophthalmic industry produces highly diluted oil-in-water emulsions with a view to patient acceptance and comfort.

Producing large volumes of diluted ophthalmic oil-in-water emulsions requires significant investment in capital equipment, and is both time and energy consuming. Emulsifying large batch sizes requires high energy input, as only a small amount of the invested energy is actually used to emulsify the oil, the major part of the energy being dissipated in the large volume of aqueous phase as heat. Moreover, yields of emulsification are generally not optimal.

For large volumes, this above-described suboptimal process results in tedious and long emulsification procedures with a potential negative impact on the chemical stability of emulsion components.

Furthermore, synthetic emulsifiers are generally required to facilitate the emulsification process. These are in common use in the food and chemical industries, but very few are FDA approved for use in products designed and approved for drug products used in the practice of ophthalmology to treat or prevent eye diseases and conditions, including dry eye issues, macular degeneration, glaucoma, diabetic retinopathy, myopia, etc. The process for approving new chemical agents of whatever kind (emulsifiers, active drugs, etc.) is tortuous, slow to achieve due to the scope and extent of testing necessary to demonstrate safety and efficacy leading to FDA approval and ultimately very costly as a result.

Therefore, there is a strong need for new approaches to enhance the emulsification steps while preparing oil-in-water emulsions for ophthalmic applications. Particularly desirable is the development of processes for preparation that overcome the above-mentioned problems and limitations associated with currently used processes. Novel features and properties of key materials utilized in the compositions is are also revealed and are the basis for the innovation disclosed in this provisional patent application.

Therefore, there is a strong need for new approaches to enhance the emulsification steps while preparing oil-in-water emulsions for ophthalmic applications. Particularly desirable is the development of processes for preparation that overcome the above-mentioned problems and limitations associated with currently used processes. Novel features and properties of key materials utilized in the compositions is are also revealed and are the basis for the innovation disclosed in this provisional patent application.

An essential and unanticipated in prior art aspect of the invention described herein is the combination of anti-oxidants that stabilizes the formulations by preventing oxidation of the catechol amino acid, Levodopa, or its prodrugs (MethylDOPA, Etilevodopa, and others that may exist.) and the catechol decarboxylation inhibitor, Caridopa protecting them from oxidative degradation preserving the formulations for use as therapeutic compositions to treat age-related macular degeneration, myopia, and diabetic retinopathy. The importance of this discovery will become apparent as this is revealed in this document.

The present invention relates to both an improved strategy for the preparation of ready-to-dilute oil-in-water emulsions including the emulsification steps, and use of already FDA approved and bio-functional materials as emulsifiers. Also, methods of preparation are provided that can be performed using equipment suitable for small to medium production lot sizes and the materials can be formulated in bulk and stored for later use before the emulsification steps are performed. Compared to currently available processes, the methods provided herein exhibit high yields, require limited amounts of energy, and reasonable processing times. More specifically, the present invention relates to processes for manufacturing ophthalmic oil-in-water micro and nanoemulsions or submicron emulsions. Such processes generally comprise steps of: (1) individually producing concentrates of both the Oil and Water phases of the resulting emulsion, both of which can be stored for later use; 2) Precisely metering the Oil Phase components into the pre-made Water Phase with gentile or high shear rate agitation, as required to produce a concentrated emulsion of the needed micelle size range suitable for use as a topically applied drop that is readily transported in the eye as required for effective treatments The emulsions can also be stored or further processed in due course that includes dilution with the required amount of water to produce an emulsion at the desired low % solids. The sterilization of the emulsion can be done after this step as part of the packaging process to complete the manufacturing by existing processes including but not limited to flash heating or radiation sterilization.

According to an embodiment of the invention, the pre-concentrates are in their individual forms, which are preferentially: 1) Bulk Oil Phase containing a non-ionic emulsifier in the mixture also including all the oil-miscible and soluble active components; 2) Bulk Water Phase containing water soluble components that can include active ingredients that can be non-ionic, or ionic in chemical nature. The emulsion concentrate is ultimately produced from the combination of 1) and 2) and is generated on-demand leading to further dilution and packaging or for temporary storage before packaging that can be done last. Processes according to the present invention aim at manufacturing thermodynamically stable oil-in-water nano or microemulsions containing dispersed droplets, preferably having a mean size generally of more than about 10 nm and less than about 500 nm. For example, the mean size of the droplets may be of more than about 10 nm and less than about 300 nm, preferably less than about 200 nm. The micelle size of the emulsions can be precisely determined by known light scattering analytical methods. In certain preferred embodiments, microemulsions or sub-microemulsions obtained using a process of the present invention are stable over periods of time in excess of 12 months when stored under specific conditions near or below room temperature by greater than Zero (0) degrees Centigrade to avoid freezing. In another aspect, the present invention relates to processes for manufacturing pre-concentrates of ophthalmic oil-in-water emulsions, preferably of ophthalmic oil-in-water microemulsions or sub-microemulsions. Such processes generally comprise a step of emulsifying an oil phase with an aqueous phase and at least one surface active agent to obtain a pre-concentrate of an oil-in-water emulsion. A pre-concentrate prepared by such a process generally has a content in oil that is higher than the content in oil of the final oil-in-water emulsion prepared by dilution of the pre-concentrate.

According to one embodiment, a pre-concentrate of the present invention is not suitable for direct application to a patient but becomes suitable for ophthalmic use after dilution to isotonic or physiologically compatible concentrations.

More specifically, in processes of the present invention, a pre-concentrate of a desired oil-in-water emulsion is produced by emulsifying an oil phase that may comprise at least one oil that is suitable for ophthalmic use, with an aqueous phase and at least one non-ionic surfactant that helps facilitate the self-emulsification process and is—may also be a natural product with functional properties that may include anti-oxidant function, for example. Oils that are suitable for ophthalmic use include, for example, olive oil, castor oil, MCT, vegetable oils, mineral oils, Omega-3 oils and any combinations of these oils that are suitable for administration to the eye.

In certain embodiments, the average hydrophilic-lipophilic balance (HLB) of the surface-active agent(s) is advantageously substantially equal to the HLB or average HLB emulsion requirement of the oil or oils of the oil phase used in the process. In certain embodiments, the oil phase may comprise one or more pharmaceutically active substances, including prodrugs. For example, pharmaceutically active substances may be selected from the group consisting of antibiotics, antiviral agents, antifungals, intraocular pressure lowering agents, anti-inflammatory agents, steroids, anti-allergic compounds, anti-angiogenic compounds, biological agents, immunomodulating agents, cytostatics, antioxidants, UV-filter compounds, fatty acids, prostaglandines and the like.

An advantage of the processes provided by the present invention is that they allow production of large volumes of emulsions without having to scale-up the emulsifying step. In another aspect, the present invention relates to processes for preparing a desired ophthalmic oil-in-water emulsion by diluting a pre-concentrate obtained as described above. Such processes include diluting one volume of a pre-concentrate with 2 to 50 volumes of a diluting aqueous solution, such that the resulting ophthalmic oil-in-water emulsion has a content in oil of 5% v/v or less, preferably of 3% v/v or less, more preferably of 2% v/v or less, even more preferably of 1% v/v or less. Preferably, dilution is performed using 2 volumes of diluting aqueous solution for 1 volume of the pre-concentrate. More preferably, dilution is performed using 10 volumes of diluting aqueous solution for 1 volume of the pre-concentrate. One advantage of such inventive processes is that oil-in-water emulsions obtained by dilution of pre concentrates are formed with a lower energy input than that required in currently used processes.

In certain embodiments, the diluting aqueous solution comprises surfactants and/or additives, e.g., tonicity agents, viscosifying agents (e.g., inert, water-soluble polymers-including Poloxamers-(nonionic triblock copolymers composed of a central hydrophobic chain of polyoxypropylene-poly(propylene oxide)) flanked by two hydrophilic chains of polyoxyethylene oxide)), polysaccharides (including Xanthan), hyaluronic acid (or its sodium salt), chitosan, buffering agents, preservatives, antioxidants, colorants or a micellar solution. Alternatively, or additionally, the diluting aqueous solution comprises benzalkonium chloride. Alternatively, or additionally, the diluting aqueous solution comprises one or more water-soluble therapeutic agents.

In another aspect, the present invention provides pre-concentrates of ophthalmic oil-in-water emulsions prepared by an inventive process. In the context of the present invention, a pre-concentrate is defined as an emulsion which may form a diluted emulsion by dilution which may form an emulsion (e.g., a microemulsion or sub-microemulsion, which is included within the term ‘microemulsion’ in the practice of the present invention, unless specifically excluded) by dilution in an aqueous medium, preferably on dilution of 1:1 to 1:50 (v/v), more preferably on dilution of 1:2 to 1:10 (v:v). Thus, a pre-concentrate may be in a liquid form or in a gel form, or in any form suitable in view of its further dilution in an aqueous medium. A pre-concentrate in the meaning of this invention is an emulsion comprising a content in oil higher than the content in oil of the final ophthalmic oil-in-water emulsion.

In certain embodiments, a pre-concentrate of an ophthalmic oil-in-water emulsion is not suitable for administration to a patient as stated earlier, requiring dilution to certain physiologically-acceptable embodiments, a pre-concentrate of the present invention comprises droplets having a size substantially equal to the size to the final ophthalmic oil-in-water emulsion droplet size, e.g., greater than about 10 nm and less than about 500 nm, less than about 300 nm, or preferably less than about 200 nm, preferably less than about 150 nm, typically less than about 100 nm as determined by light scattering analysis.

In certain embodiments, a pre-concentrate of the present invention is stable for periods of time in excess of 24 hours, preferably in excess of 3 days, more preferably in excess of 7 days.

In another aspect, the present invention provides ophthalmic oil-in-water emulsions obtained by dilution of a pre-concentrate as described herein.

In another aspect, the present invention provides pharmaceutical compositions comprising an effective amount of an oil-in-water emulsion prepared according to an inventive process disclosed herein.

In yet another aspect, the present invention relates to the use of an oil-in-water emulsion prepared according to an inventive process disclosed herein for the manufacture of a medicament for the treatment of an eye disease or condition.

In a related aspect, the present invention also relates to medicament comprising an oil-in-water emulsion prepared according to an inventive process disclosed herein.

In still another aspect, the present invention provides methods for the treatment of an eye disease or condition in a subject. Such methods generally comprise a step of administering to the subject an effective amount of an ophthalmic oil-in-water emulsion prepared by an inventive process described herein. Administration may be by topical, intraocular or periocular routes. Such methods may be used to treat any eye disease or condition, for example, glaucoma, ocular inflammatory conditions such as keratitis, uveitis, intra-ocular inflammation, allergy, and dry eye syndrome ocular infections, ocular allergies, ocular infections, cancerous growth, neo-vessel growth originating from the cornea, dry eye disease, cataracts, retinal oedema, macular oedema, diabetic retinopathy, retinopathy of prematurity, degenerative diseases of the retina (macular degeneration, retinal dystrophies), and retinal diseases associated with glial proliferation or for healing trauma or chemical injuries.

These and other aspects, advantages and features of the present invention will become apparent to those of ordinary skill in the art having read the following detailed description of the preferred embodiments. Need: Eye Drop to Treat Eye

### Diseases, Including Macular Degeneration

Age-related macular degeneration (AMD) is a common cause of blindness in developed countries, with increasing prevalence as life expectancy increases. The disease is characterized by degeneration o the central part of the retina, the macula, which is responsible for high acuity vision. In the US, greater than 15% of he population past the age of 70 has AMD, for which we lack adequate prevention or treatment.

Neovascular AMD (nAMD) is characterized by the abnormal ingrowth of new blood vessels from the choriocillaris into the sub-pigment epithelial or subretinal space, which can cause fluid and blood to leak.

The observed angiogenesis is due to excessive vascular endothelial growth factor (VEGF). nAMD is a much less common type of AMD, representing only 10-15% of all AMD cases, however, it accounts for 90% of the vision loss from AMD.

In 2008, the Mckay et al. (supra), a de-orphaned receptor on the surface of human retinal pigment epithelial cells GRP143. It was discovered that the ligand for this G-protein coupled receptor was levodopa (L-DOPA), an intermediate in pigmentation. GPR143 is found on the apical surface of the RPE, a monolayer of pigmented cells located between the choroid and neural retina, likely the primary issue that initiates AMD pathobiology. These RPE cells release VEGF in response to anoxia and other stimuli; excess VEGF release is believed to initiate and cause nAMD. In RPE cells, activation of GPR143 by levodopa significantly upregulates pigment-epithelium derived factor (PEDF), a potent anti-angiogenesis factor, and simultaneously downregulates the production of VEGF. VEGF is the target of current therapies to low angiogenesis. This strategy is successful, but requires frequent intravitreal injections.

The inventors speculated that Levodopa therapy could decrease the development of AMD and nAMD. In 2016, Brilliant et al. (Brilliant, N. et al, Mining Retrospective Data for Virtual Perspective: Drug Repurposing-L-DOPA and Age-Related Macular Degeneration; Amjmed (2016), doi: 10.1016/j.amjmed.2015,10.1015) performed a retrospective analysis to determine if there was a possible link between Levodopa and AMD. They examined clinical data encompassing greater than 87 million individuals. Their-study showed that individuals with a history of Levodopa prescriptions are significantly less likely to develop any type of AMD. Further, if they did develop AMD, the age of onset was significantly delayed by seven years. They also found a similar delay for nAMD, and a decreased odds ratio of 0.65. More recently, others have shown in a prospective proof of concept study that treatment of patients with new onset nAMD, naïve to anti-VEGF with Levodopa resulted in decreased intraretinal fluid and improved vision with 1 month. In those with continued Levodopa therapy, there was a decreased need for anti-VEGF injections compared to historical controls.

They concluded that using levodopa as adjuvant therapy for nAMD could well alter the course of nAMD pathogenesis and save billions of dollars without sacrificing vision.

There is now substantial laboratory and epidemiological evidence that Levodopa has a positive role in preventing or delaying AMD and its progression to nAMD. There is also a proof-of-concept clinical trial that shows Levodopa positively affects nAMD. We have thus developed a formulation for a Levodopa-containing drop to treat, prevent, or delay the progression of AMD and nAMD.

There are benefits to topical delivery of AMD therapeutics. The application of a drop can be done by the patient without the need for a doctor visit and possible injection injections. The delivery of a drop targets delivery to the “end organ” and minimizes systemic toxicity and side effects from interference with other organ systems. Topical delivery also allows treatment of each eye independently. where one can be treated more aggressively with advancing disease or one randomized and treated with a placebo for clinical trials. Topical therapy is the mainstay for ophthalmology and ophthalmologists have used for prescribing for a variety of drug cases. Patients are also likely to have a more favorable opinion on topical drops vs intravitreal or systemic medication. Finally, the topical formulations become new drug compositions with attendant patent lives.

### Inventive Compositions and Processes for Their Preparation:

Inventive formulations and the self-emulsification processes for producing them will be described in principle and in detail in this section. The inventive process for preparing the emulsified formulations of this invention require emulsifying agents, e.g., surfactants, having specific properties suited to the process of self emulsification. These can include ionic or non-ionic surfactants. However, non-ionic surfactants are generally referred as they are compatible for use with a wide variety of emulsifiable components whereas ionic surfactants generally have more limited utility for components that are both ionic and non-ionic in their chemical nature. There are two preferred classes of non-ionic surfactants: 1) polysorbates including polysorbate 80 and the 2) Polyoxamers-block copolymers of polypropylene oxide and ethylene oxide that are thermally reversible in that they become viscous as temperature rises near normal body temperatures. Polyoxamers are sold under the Pluronic™ polymer trademark by BASF.

Pluronics® polymers constitute a family of amphiphilic block copolymers, broadly used in pharmaceutical and clinical applications due to their low immunogenicity and exceptional biocompatibility. Certain Pluronics® polymers have been approved by the U.S. Food and Drug Administration (FDA) and the European Medicines Agency (EMA) for their use as surfactants, solubilizers, emulsifying agents and absorption enhancers. In addition, they are employed as matrices and carriers, according to their most characteristic properties: temperature-dependent gelation and micellization. These properties enable the transport of active pharmaceutical ingredients (APIs), especially those ingredients with poor water solubility. Under certain conditions, Pluronics self-assemble into different supramolecular structures, where nanosized micelles stand out. Micelles are defined as colloidal NPs with a core-shell architecture and sizes in the range of 10-100 nm. The amphiphilic unimers of Pluronic polymers self-assemble at critical micelle concentrations (CMC) and form either direct micelles (DMs) or reverse micelles (RMs) depending on the external solvent. In general, polymeric micelles are outstanding drug delivery vehicles with three unmatched advantages.

Firstly, their relatively small size, which favors blood circulation, tissue penetration, cell uptake and in vivo performance of encapsulated drugs, as the preferred size range for drug delivery is 40-50 nm. Secondly, the polymer structure regulates the drug release preventing a rapid clearance, prolonging its circulation time and decreasing the toxicity of the loaded drug. Thirdly is the feasibility of large-scale manufacturing, as it is the simplest assembled entity with a well-defined molecular structure. These advantages promoted the global acceptance of micelles as a first-line drug formulation technology. Beyond the capacity of Pluronics polymers to form micellar nanocarriers, these copolymers have been used in the modification of a myriad of other NPs, generating hybrid structures with mixed properties: polymeric NPs, metal NPs, nano-suspensions, liposomes, dendrimers, nanogels, polymeric micelles and solid lipid NPs. Furthermore, they have been functionalized with different moieties such as pH-sensitive, biological-responsive moieties, antibodies, aptamers, folic acid, or drugs. However, only a handful of Pluronics® polymer formulations are in clinical research since these systems show some limitations, mainly their complex characterization, instability under physiological conditions and low loading efficacy. Furthermore, the broad variability of Pluronic polymers requires a thorough study on their toxicity aspects. It has been reported that Pluronics introduced in the body via routes other than dermal exposure have a rapid clearance from the body, thus reducing their potential toxicity. According to the Clinical Toxicology of Commercial Products, most Pluronics® polymers are at the lower limit of the “slightly toxic” class, with high values around 5 g/kg. Considering their surfactant nature, the toxicity decreases as the ethylene oxide: propylene oxide (EO): (PO) ratio increases and as the molecular weight of the hydrophobic part increases. In particular, the most widespread member, Pluronic® F127, is described as the least toxic member, but can also produce undesirable effects in animals such as hypertriglyceridemia and hypercholesterolemia. Nevertheless, most studies in humans indicate that Pluronics are safe to be used.

### Physicochemical Properties of Pluronics and Self-Assembly Behavior

Pluronics® polymers are synthetic, triblock copolymers composed of ethylene oxide (EO) and propylene oxide (PO) repeating units. The most common copolymers, so-called “normal” Pluronics, exhibit a general formula (PEO) x-(PPO)y-(PEO) z, being hydrophilic poly(ethylene oxide) (PEO) chains on the sides and a central hydrophobic poly(propylene oxide) (PPO) chain (x, z and y are the respective numbers of PEO and PPO moieties in the polymer chain, and may be the same or different. However, reverse analogues (Pluronic-R) are also described with a general formula (PPO) x-(PEO) y-(PPO) x, exhibiting a central hydrophilic chain flanked with two hydrophobic chain. Normal Pluronics are commercially known as Pluronics® polymers (manufactured by BASF, Ludwigshafen, Germany) or Poloxamers® (manufactured by ICI, Tokyo, Japan) while reverse Pluronics are named Pluronics-R (produced by BASF). The nomenclature employed for these polymers—a letter followed by two or three digits-indicates their structural differences and differs depending on the supplier. Poloxamers are named using the letter P and three digits: the first two digits represent the molecular weight of the PPO chain, multiplied by 100; the last digit means the PEO percentage in the copolymer multiplied by 10. For Pluronics® polymers, the letter indicates the physical state of the product (P=paste; L=liquid; F=flakes); the first and/or middle digits indicate the molecular weight of the PPO block multiplied by 300; the last digit gives the PEO percentage multiplied by 10. For example, Poloxamer P188 or Pluronic L61 is a copolymer build-up with a PPO molecular mass of 1800 g/mol and 80% of PEO. These polymers are non-ionic amphiphiles and exhibit an extraordinary versatility arising from the multiple combinations of molecular weights and commercially available PEO: PPO ratios. These lead to a variety of physicochemical and biological properties, which have been broadly explored for normal Pluronics but less studied for the reverse counterparts. In solution, Pluronic chains spontaneously form thermodynamically stable supramolecular structures, including micelles, gels, rod-like structures, and lamellar phases. The polymer composition, concentration and temperature will determine the resultant structure, among other variables. In general, at low concentrations, Pluronic solutions contain individual molecules that aggregate in spherical or rod-like micelles above the CMC, which can further form lyotropic liquid crystals at higher concentrations. Micellization phenomena occurs around the CMC, but above the CMC the micelles may arrange in a lattice structure (gel). The Pluronic concentration at which gelification phenomena occurs is called as critical gel concentration (CGC). The micellization process of Pluronics® polymers are entropy-driven and endothermic, and it is a less favorable process for reverse Pluronics® in water. For normal Pluronics®, direct micelles (DMs, in oil in water emulsions) are obtained when a hydrophilic external solvent is used, while reverse micelles (RMs, in water in oil emulsions) are formed in a lipophilic environment. In the past, the term “reverse micelle” was restricted to systems with a low water content, but it is common to use the term for micellar systems in a predominantly organic media.

These reversible polymeric structures are formed as a result of a delicate balance between strong covalent bonds that hold the PEO and PPO blocks together within each Pluronic unimer and the reversible intermolecular forces that assemble them. The micelles' size is dictated by different parameters such as the molecular weight of the copolymer, the aggregation number (Nagg) of the amphiphiles, the ratio of hydrophilic and hydrophobic chains, the amount of solvent trapped inside the micellar core and the preparation process. In solution, Pluronic chains spontaneously form thermodynamically stable supramolecular structures, including micelles, gels, rod-like structures, and lamellar phases. The polymer composition, concentration and temperature will determine the resultant structure, among other variables. In general, at low concentrations, Pluronic solutions contain individual molecules that aggregate in spherical or rod-like micelles above the CMC, which can further form lyotropic liquid crystals at higher concentrations. Micellization phenomena occurs around the CMC, but above the CMC the micelles may arrange in a lattice structure (gel). The Pluronic concentration at which gelification phenomena occurs is called as critical gel concentration (CGC). The micellization process of Pluronics is entropy-driven and endothermic, and it is a less favorable process for reverse Pluronics® polymers in water. For normal Pluronics® polymers, direct micelles (DMs, in oil in water emulsions) are obtained when a hydrophilic external solvent is used, while reverse micelles (RMs, in water in oil emulsions) are formed in a lipophilic environment. In the past, the term “reverse micelle” was restricted to systems with a low water content, but it is common to use the term for micellar systems in a predominantly organic media. These reversible polymeric structures are formed as a result of a delicate balance between strong covalent bonds that hold the PEO and PPO blocks together within each Pluronic unimer and the reversible intermolecular forces that assemble them. The micelles' size is dictated by different parameters such as the molecular weight of the copolymer, the aggregation number (Nagg) of the amphiphiles, the ratio of hydrophilic and hydrophobic chains, the amount of solvent trapped inside the micellar core and the preparation process.

Other parameters include the enthalpy of micellization (AHmic); the Nagg, which indicates the number of surfactant unimers in a spherical micelle; and the hydration number, defined as the number of water molecules moving with a micelle as a kinetic entity. The amphiphilic nature of a surfactant, which is crucial for the micellization capacity, is described by the hydrophilic-lipophilic balance (HLB). The HLB is calculated from the weight percentage of the hydrophilic groups to the hydrophobic groups in a molecule, with values ranging from 1-20. This parameter suggests the type of micelle to be formed: RM for HLB values <10 (predominantly hydrophobic surfactants) and DM for HLB values >10 (predominantly hydrophilic surfactants). The HLB value is also related to the particle size, drug loading, stability, and drug release profile. Considering the HLB value and PPO chain length (“y” value in (PEO) x-(PPO) y-(PEO) x), Pluronics® can be divided into four types: type I, “hydrophilic” Pluronics® polymers with HLB 20-29, such as F68, F108 and F127; type II, with HLB<20 and y >2 y<30, such as L35, L44 and L64; type III, with HLB<20 and y=30-60, such as P85, P105 and L61; and type IV, with HLB<20 and y >60, such as P123 and L121. Polymers with higher HLB values are less prone to form micelles. Taking into account that Pluronic micelles are typically employed as carriers of poorly water-soluble drugs in a water aqueous solution, hydrophobic Pluronics (with low HLB) could improve their loading capacity; nevertheless, these parameters also decrease the colloidal stability of micellar systems, exhibiting high CMC values, which justify that most examples in the literature employ hydrophilic Pluronics. To characterize the cargo loading within micelles, other parameters are used such as the drug loading degree (LD), the drug encapsulation efficiency (EE), the micelle/water partition coefficient (P) and the standard free energy of solubilization (ΔGO).

In the practice of the present invention, hydrophilic Pluronics® polymers are preferred, but may be blended with a minor proportion of intermediate, and slightly more oleophilic Pluronics® polymers (e.g., with the hydrophobic group y>16 y <26).

These options are useful for various applications including topical use. The hydrophilic-lipophilic balance of a surfactant is a measure of the degree for which it is hydrophilic or lipophilic, determined by calculating values for the different regions of the molecule.

Pharmaceutical excipients are included as potential ingredients for the compositions described in this document. The phrase “functional excipient” usually implies that the excipient has specific functionality such as—carboxyl,-hydroxyl,-amino,-thiol, etc. For purposes of this filing, it may also mean that the excipient may have additional functional characteristics in that the compound participates as an active component of a biochemical pathway as a solvent, co-factor, transporter, receptor agonist or antagonist, an enzyme, as a catalyst, an electron transfer reducing or oxidizing agent, as a eutectic agent, an anti-microbial or anti-viral agent, a photosensitizer, a UV absorber, or other class of compounds having specific features important to the process of manufacture and/or to the targeted therapeutic characteristics of the formulation. Functional excipients can be natural products, obtained from plants, or other living species (fungi, insects, algae including microalgae) that could include, for example, enzymes such as protease or kinase enzymes.

A focus of this invention is to restore systemic redox dysfunction and imbalance, a root cause of ocular diseases including macular degeneration and diabetic retinopathy and others that will be explained in the next section.

### Redox Balancing and Oxidative Homeostasis

Redox homeostasis is a delicate balancing act of maintaining appropriate levels of antioxidant defense mechanisms and reactive oxidizing oxygen and nitrogen species. Any disruption of this balance leads to oxidative stress, which is a key pathogenic factor in several ocular diseases. The current evidence for oxidative stress and mitochondrial dysfunction in conditions affecting both the anterior segment (e.g., dry eye disease, keratoconus, cataract) and posterior segment (age-related macular degeneration, proliferative vitreoretinopathy, diabetic retinopathy, glaucoma) of the human eye.

The human eye is not resistant to processes associated with aging. At the front of the eye, the cornea and lens are particularly susceptible to oxidative stress that can be attributed to direct exposure to ultraviolet (UV) light emitted by the sun. As a highly metabolic tissue, the retina is also at increased risk of age-associated processes that contribute to increased oxidative stress and neurodegeneration. Damage to these cell layers may cause deficits in visual acuity or progressive vision loss and significantly affect mobility and quality of life.

A homeostatic balance of antioxidants and reactive oxygen species (ROS) is required for the healthy functioning of ocular tissues. An imbalance of antixodants (AOX) and ROS through the depletion of antioxidants or excessive ROS accumulation will result in oxidative stress and drive subsequent disease progression.

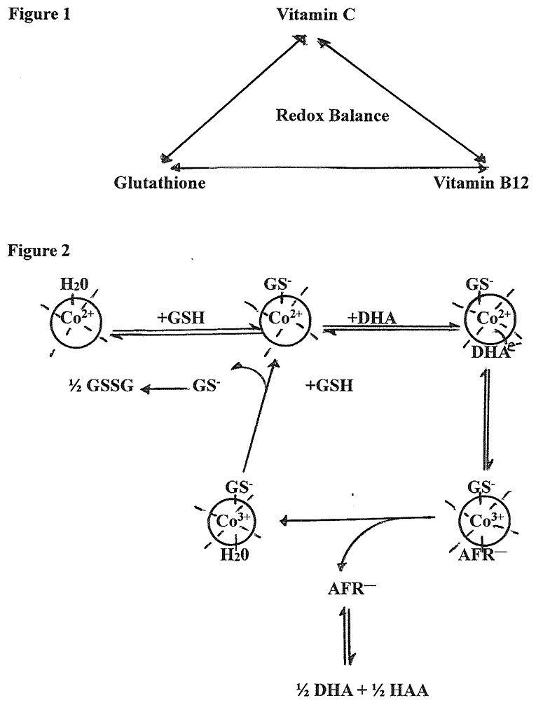

The relationship between Vitamin C (ascorbic acid), glutathione, and Vitamin B12 has been the subject of in-depth research for many years. Recent studies have identified that aquahydroxocobinamine (hydrated Vitamin B12) acts as a co-factor to convert the two-electron oxidized form of Vitamin C, dehydroascorbic acid (DHA) to ascorbic acid by glutathione (GSH). The synergy between these agents to protect cells (including those in the ocular system) from oxidative degradation, and the significance of re-establishing redox balance in the process is highlighted in FIG. 1.

FIG. 1: Balancing the oxidation-reduction (redox) system at the cellular level requires several antioxidants involved in regulating multi-genetic pathways to maintain redox homoeostasis. The combination of Vitamin C, Glutathione, and Vitamin B12 (a co-factor required for GSH synthesis in-vivo is important to meet this objective.

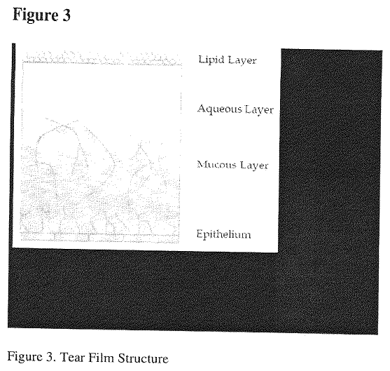

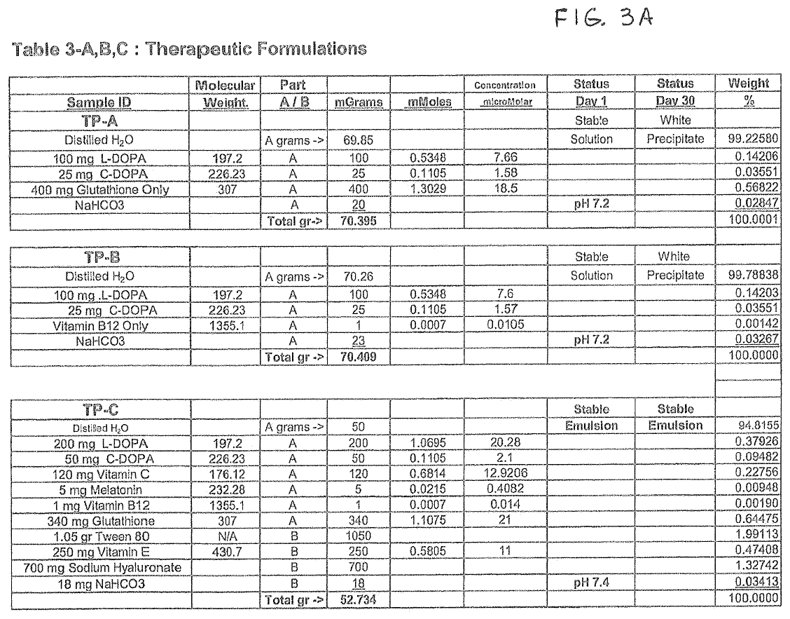

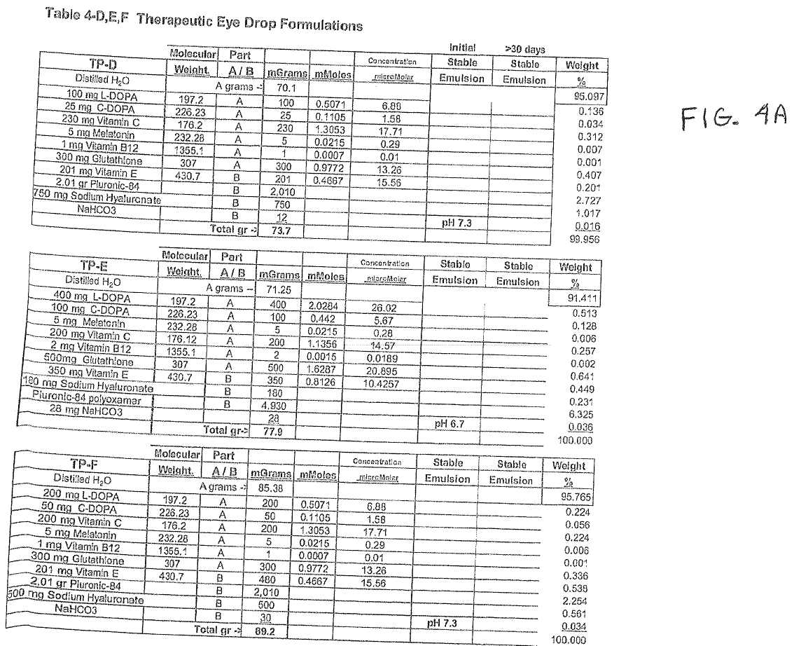

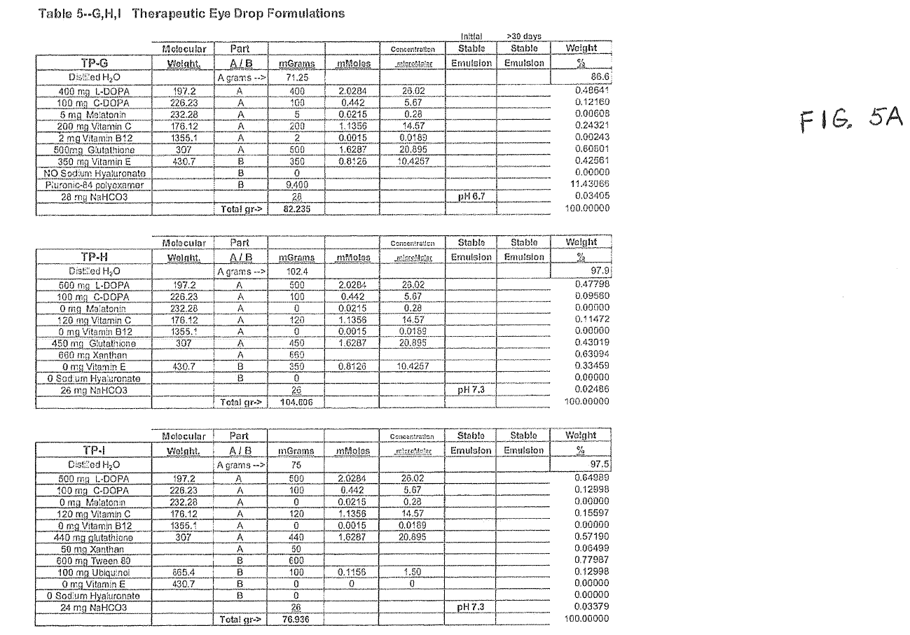

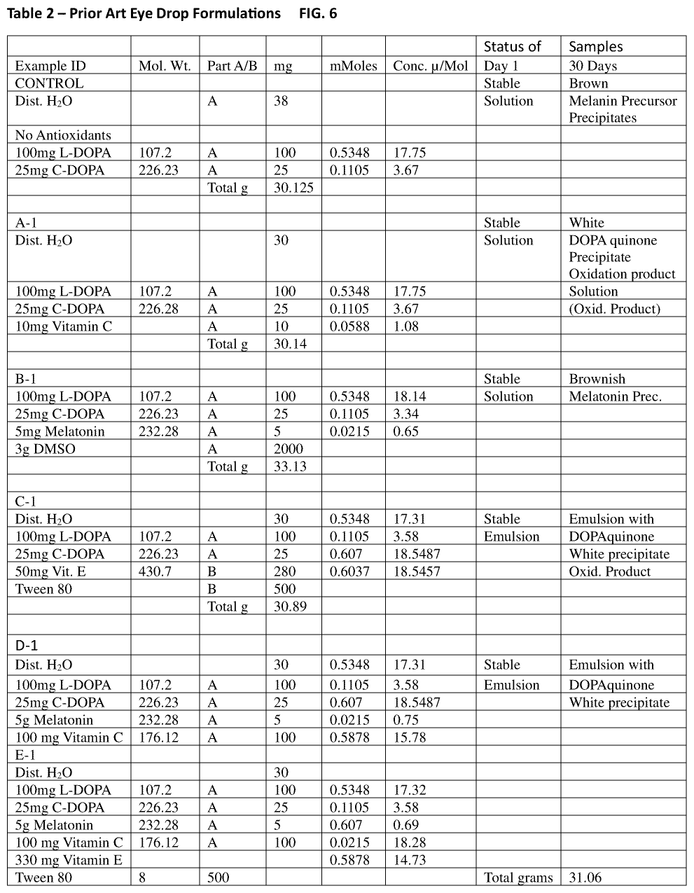

Stabilizing the eye drop formulations while at the same time restoring redox balance in ocular tissues after topical application is therefore a critical focus of the invention. Ascorbic acid is an electron donor (possesses an intensive reductive potential). It can be oxidized to the ascorbic radical and then to dehydroascorbic acid. Ascorbic radical is a relatively stable and unreactive molecule. On the other hand, dehydroascorbic acid is an unstable molecule with a short half-life, and it is rapidly reduced back to ascorbic acid by glutathion dependent cellular reducing systems. The biochemistry is detailed in FIG. 2.

FIG. 2 shows how the Ascorbic Acid (HAA)/Glutathione/Vitamin B12 Redox Cycle Vitamin B12 controls the synthesis of anti-inflammatory cytokines and growth factors, reducing systemic inflammation. Ascorbyl Free Radical (AFR—) n Dihydryoascorbic acid (DHA) are shown in the system of the Redox Cycle.

### Drug Transport in Ocular Tissues

The eye is a complex organ divided into anterior and posterior segments. The anterior segment consists of the cornea, conjunctiva, iris, pupil, ciliary body, anterior chamber aqueous humor, trabecular meshwork, the lens while the posterior segment consists of the vitreous humor, sclera, retina, choroids, macula and optic nerve.

Topical application of therapeutic formulations targeting the posterior segment of the eye of necessity must traverse portions of both the anterior and posterior segments to arrive at the target tissue. In the case of age-related macular degeneration and diabetic retinopathy, the target tissues are primarily in the retina and specific cells including the retinal pigment epithelium (RPE) where GPR143, the receptor that L-DOPA binds to via ligation. Ligation with L-DOPA leads to gene expression of a specific protein-neurotrophic factor, PEDF. The greatest challenge in the treatment of eye diseases is the short precorneal retention time of locally applied drugs on the surface of the eye and poor ocular availability, which is less than 5%. The tear film, which covers the tissues of the ocular surface, is composed of an outer lipid layer and an inner aqueous layer. This aqueous layer is comprised mainly of water and mucins, which are high molecular weight glycoproteins. Throughout the body, the function of mucins, or the mucus barrier, is to protect cellular surfaces and maintain water balance, and this is also the functions of mucins on the ocular surface. In the tear film, the mucus barrier is comprised of membranes associated mucins, which form a dense layer near the corneal epithelium, and secreted mucins, which form an outer layer and are less densely arrayed. Secreted mucins, the first line of defense in the mucous barrier, move within the tear film and bind to foreign particles, including allergens and pathogens. The secreted mucins, with associated particles, and tear film are moved out to the nasolacrimal duct during blinking to rapidly clear the ocular surface.

The ability of polymers to initiate non-covalent interactions with mucin enables their adherence to the corneal and conjunctival surfaces of the eye. This property of the polymer enables longer retention of the applied liquid preparation on the surface of the eye, less loss due to drainage, better penetration of drugs, or longer local activity.

Mucus is an adhesive viscoelastic gel which covers most mucosal surfaces in the body. The main roles of the mucus layer are to maintain a healthy wet surface and to form a barrier against various pathogens, drugs, and other environmental toxic agent. The mucus is composed from water (<95%, w/w), extracellular glycoproteins, lipids such as fatty acids, phospholipids, salts (˜1%, w/w), carbohydrates, cholesterol, defensive proteins (i.e., lysosomes, defensins, trefoil factors, etc.) and mucin (<5%, w/w). Mucins are large extracellular glycoproteins with molecular weights from 0.2 to over 50 MDa that are negatively charged due to the presence of terminal sialic acid (pKa of 2.6) and sulphate groups. The bonding between layers is non-valent, and primarily ionic in nature.

Mucoadhesion is defined as the adhesion of a natural or synthetic polymer to a mucous membrane through physical or chemical interaction. The phenomenon of mucoadhesion is usually explained through two successive phases. In the first phase, contact is established between the mucoadhesive polymer and the biological substrate (in this phase, the ability of the polymer to spread over the biological substrate and interfacial forces play a key role). As the topically applied drops are assimilated into the surface spreading uniformly over the anterior segment, the adhesion to the mucous layer is an important factor in drug deliver. Higher adhesion translates to increased drug delivery with a longer retention time of the drug at this interface. While it is possible to produce formulations without mucoadhesive polymer additives, their efficacy in delivery of the therapeutic components is limited, that ultimately also affects compliance as these polymers also increase comfort.

The preferred mucoadhesive polymers for the formulations detailed in this Application are 1) hyaluronic acid and its sodium salts that are anionic charged; 2) xanthan, a naturally occurring polysaccharide that can be produced synthetically that is also anionically charged; 3) chitosan, positively charged biocompatible biopolymer with low-toxicity. Hyaluronic acid salts and xanthan are water soluble, and therefore readily dispersed/dissolved in aqueous eye drop formulations. Chitosan, however, has very poor, almost zero, water solubility except in strongly acidic media with a pH of 6 or less. While it is preferred due to its cationic charge that increases adhesion to mucus, rendering its use is problematic without improvements in solubility and dispensability.

Incorporating the sodium hyaluronate into the eye drop as a thickener, produces formulations with micelles having a net negative charge at a neutral pH. If chitosan can be solubilized without solvents, it has a net positive charge in solution and could act as an additional thickening agent, but also shift the charge of the formulation to positive under the right conditions. Tables 3ABC, 4ABC, and 5GHI evidence details of Therapeutic Sample formulations described herein within the current specification, starting with Sample TP-C(Table 3).

An aspect of this work is to develop and define Eye Drop candidates with sufficient stability (against oxidation or other mechanisms of degradation) following production and through storage and use to be viable for use by physicians and patients. Thus the column in the tables titled “Status Day 30” provides a measure of the unique stability of these formulations and the resulting focus on these formulations as examples of the reduction to practice of an invention.

The formulations may be complex with many alternative components, that are justified in the Specification. Simplifying their complexity could become the focus of the next phases of product development. However, at present it is preferred to retain all the components as larger numbers of components are likely to become part of any product.

As for the concentration ranges it is suggested that concentrations ranging from at least about 50% up to about 200% of those described in the Examples would typically be useful for each component. For specific compounds (ex. Vitamin C, E), this has been calculated in units of micromolar (micromoles/liter) in the tables.

Solubilizing Insoluble Compounds Including Melatonin/Chitosan without Solvents

Melatonin is an endogenous compound produced in the body that is an anti-oxidant but also regulates the circadian rhythm at the cellular level that is operative in healthy individuals. Circadian rhythm regulates the sleep/awake cycle and is also tied to light exposure with sunlight preferred. Melatonin is practically insoluble in water, hence delivering it via a topical drop requires innovation that will be revealed in the following example.

Chitosan is a linear cationic heteropolymer consisting of D-glucosamine and N-acetyl-D-glucosame units. In the following experiment, I will generate a dilute aqueous solution of chitosan without solvents.

### EXAMPLES

### Example 1

125 milligrams of chitosan polysaccharide was added to a 250 ml Pyrex beaker, followed by 125 milligrams of Vitamin C. Next, 10 mg of melatonin (powder) was added. The beaker was equipped with a magnetic stirrer and the mixer also had a built in hot plate, as needed. Next, 50 grams of distilled water was added and the mixture was stirred at room temperature for 1 hour. At this point, over 90% of the solids had gone into solution. The temperature of the mixture was raised to 50 C to complete the dissolution of the powders and after 30 minutes, the solution was free of undissolved material and was slightly turbid. The pH of the solution was 5.8.

Next, 200 mg of Levodopa and 50 mg of Carbidopa were added, followed by 460 mg of glutathione and 1 mg of Vitamin B12. The slurry was mixed for an additional hour at which point, dissolution was complete. This solution is labeled as Part A of the eye drop formulation. The solution was then set aside for use in making eye drop formulations as will be explained in the following sections. This process demonstrates that an aqueous solution of a water insoluble compound can be produced using ascorbic acid as a dissolution aid. Melatonin contains both amide and amine groups while chitosan contains the amino group of the D-glucosamine linkage. Both the amide and the amine groups can react with the acidic proton(s) of ascorbic acid to form complexes that are water soluble and dispersible. A side benefit of these complexes are that they can carry along unreacted ascorbic acid that can function as an antioxidant in the formulation. The pH of the solution can be altered as needed for use in producing the eye drops of this invention. This strategy for dissolving insoluble compounds in water can carry over to other compounds, including melatonin that contain weak bases, including amino or amide groups. In addition, the intramolecular ascorbate in the complex can be reduced by gluatathione to recycle Vitamin C in-vivo as a side benefit.

Part B of the formulation was next prepared by adding 690 mg of Tween 80 and 180 mg Vitamin E to a 125 ml beaker containing a magnetic stirrer. Part A was then added with stirring to Part B to produce the emulsion that formed immediately. This was evident from the milky appearance generated. The pH of the emulsion at this point was unchanged from Part A by itself and remained at 5.8. Next, 28 mg of NaHCO3 was added slowly with mixing to raise the PH to 6.9. The last step is the slow addition of 500 mg sodium hyaluronate (NaHA) powder while mixing. NaHA tends to clump, but by adding it slowly clumping can be reduced. The mixing was continued at room temperature for another hour at which point the NaHA was totally dissolved and the viscosity of the formulation was raised to the desired end point so It can be used as eye drop that is easily administered to the eye surface. At this point, the formulation is stable and can be packaged or held for later packaging/distribution and use by patients or eye care specialists.

### Examples, Continued

The materials employed in the examples are all commercially available, intended for human use, unless otherwise indicated. USP pharmaceutical grade options are preferable if the formulations are intended for human use including testing.

Self-emulsifying formulations of this invention are directed for use as topically applied medicaments, including those uses in the practice of ophthalmology in treating eye disease including age-related macular degeneration, diabetic retinopathy, and myopia and others such as glaucoma, dry eye disease. They are produced from components that are generally water soluble (hydrophilic) along with others that are water insoluble (hydrophobic). Combining these two-water solubles and water insolubles-requires compositions and methodologies that produce emulsions of the oil in water type and processing that facilitates that result. In some cases, it has proven possible to produce solutions containing water insoluble components in water only by the order or addition of the components to the solution without making an emulsion. This will be explained and demonstrated by example.

Part A—the aqueous component containing water soluble or water dispersible compounds or agents, and Part B-lipophilic components that are not water soluble or readily dispersible or miscible. The intimate blending of these two main components-Part A and Part B-is essential to the ultimate functionality of the end product—for example, an eye drop or topical cream or lotion that can be utilized as a key part of an effective treatment for specific medical conditions. The most effective delivery system for such an intimate blend is an emulsion having very small micelle structures less than about 100 nanometers in dimension that is stable long term for weeks or months for use by physicians and patients. Means and compositions for producing this class of emulsions are the subject of this invention. Specifically, well known biological agents that are effective in treating the outlined medical conditions can be incorporated into these emulsions for effective delivery. Topical delivery to the diseased tissues represents a targeting strategy for treatment as opposed to systemic delivery via oral means that requires passage through the gut and then to the bloodstream and finally to the diseased tissues in order to effect a therapeutic result. While this process can be effective, it is generally not efficient as the passage through the gut and other tissues before arrival at the target tissue results in what is referred to as the 1st pass effect—a loss of the concentration and chemical composition of the active ingredients due to adverse metabolic reactions for example hydrolysis or enzymatic breakdown of chemical structures. Hence topical delivery can avoid these complications if the therapeutic components can be applied directly to the diseased tissues or be to them by transported to them by localized diffusion or transmembrane transport.

### Example Formulation Preparation:

### Prior Art Eye Drop Formulations:

In some cases, the formulations are homogenous solutions rather than emulsions dependent on the aqueous solubility and compatibility of the ingredients used in those examples. In examples that produce emulsions, due to components that are individually either water soluble or insoluble (example Vitamin E) that require emulsification. This difference can also lead to formulations with increased retention time in the tear film of the eye after delivery, to prolong the time for transport to the targeted tissues, resulting in improved therapeutic efficacy.

The self-emulsifying formulations of the present invention consist of both PART A and PART B components. PART A consists of the aqueous soluble (hydrophilic) compounds while PART B contains the lipophilic (hydrophobic) compounds that are water insoluble by nature. A key component that can be located in either PART A or PART B (before mixing) but preferably in PART B are compounds that act as emulsifying agents. The emulsifying agents are selected from those in common use in FDA approved medications and recognized as safe.

### Part A Preparation Process:

The water-soluble components of Part A are dissolved in distilled water. After dissolution is complete, the mixture is filtered through course filter paper to remove any undissolved particles (typically inert components—including excipients—of the tablet or capsule, to be dissolved for subsequent delivery). For large batches, this can be done using an Erlenmeyer flask with magnetic stir bar mixing for small batches, or with larger vessels and stirring equipment. Generally no heat is required during this step but may increase the rate of dissolution of agents to shorten the manufacturing cycle time.

### Part B Preparation Process:

The lipophilic components of the emulsion are dispensed into a vessel large enough to contain the quantity of the self-dispersing emulsion including the amount of Part A required. In the case of the inventive formulations described herein, a plastic vial capable of holding ˜50 milliliters of emulsion was used that also had a closure cap attached for storing and preserving the products for a period of time (at least one month). After all the desired components of PART B have been added to the vial (or other suitable vessel), they are carefully mixed together using a sterile device (spatula, or other depending on batch size). This step helps facilitate the self-emulsification process.

### Production of the Self-Emulsifying Emulsions:

A pre-weighed quantity of PART A was dispensed into a delivery vessel from the master batch to be combined with PART B that was prepared as described above for each formulation. At this point, the materials are in readiness for producing the self-emulsifying formulation. PART A is next slowly added to PART B with gentle agitation (stir stick, spatula, or hand motion). As the addition proceeds, the emulsification process becomes readily apparent as the resulting mixture becomes turbid resembling milk or latex paint. After the addition of PART A is complete, it is best to ensure complete emulsification by sealing the vial, and gently shaking it for a minute or so until there is no evidence of PART B that has not been emulsified is present. This is easy to observe. The final step in the process is to place the sealed vial containing the self-emulsified formulation into an ultrasonic water bath for a few minutes. This last step is optional, but helps to homogenize the emulsion and also to reduce the size of micelles, for example from the micro to the nano scale size range. This process can be quantified using a light scattering instrument capable of precisely measuring the size of micelles. The formulation components for the inventive formulations are described in Table 1 while the individual formulations are described in detail in Table 2.

An example of a solution eye drop which is not an emulsion, incorporating water insoluble melatonin, is detailed in Table 6 (Prior Art formulations). In order to dissolve it, a small amount of DMSO solvent was added. Adding a solvent for it like DMSO would be required. To demonstrate that melatonin by itself is not effective in protecting the L-DOPA/C-DOPA components from oxidation, I incorporated sample B-1 (Table 6). In Sample D-1, dissolving melatonin was accomplished without DMSO by using Vitamin C as a component of the formulation. Since Vitamin C (in solution) is a weak acid, it is able to dissolve melatonin—a weak base, and keep it in solution even though it is ostensibly not water soluble. This is pH dependent in that the solution must remain slightly acid with a pH of 6.8 or so maximum. This is further Exemplified in sample TP-3, Table 3, sample TP-C, and Table 4, samples TP-D, TP-E, and TP-F where melatonin has been formulated into Part A—the aqueous part of the emulsions and remains in solution after emulsification.

Note that some of the formulations exemplified are not emulsions but homogeneous solutions where the components are all water soluble. The order of addition in the manufacturing process may be critical in these cases, but will be explained in the detailed descriptions.

The components of the formulations are described in detail in the following section are detailed in the tables present as Table 1.

Formulations representing prior art eye drop compositions are described in Table 6, including a NON-PRIOR ART control sample with L-DOPA/C-DOPA with no antioxidants. Note that at 30 days without antioxidants L-DOPA and C-DOPA have oxidized to a brown precipitate that approximates melanin in composition.

### Commentary on the Examples:

The examples are illustrative of the novel compositions, with possible options of components listed in Table 1 and the combinations we claim as unique. They are not to be considered as limiting in any way of the concentrations or ratios of those components within reason. For Example no individual component (exclusive of water) can be more than say ˜50% of the total mass of the components. For use in treating patients, eye drops are diluted to concentrations referred to as “isotonic” or “hypotonic” in that the water content of the formulation is comparable or lower than the tissues being contacted. This will facilitate the drug transport process without challenging the water content of the affected tissues. The most effective actinic agent concentrations of the formulation components must be determined via clinical trials focused on efficacy and cannot be precisely predicted in advance.

The detailed examples provided have potential uses as topical treatments for selected eye diseases, including but not limited to neovascular macular degeneration, diabetic retinopathy, myopia, dry (non-vascular) macular degeneration, and other eye diseases as outlined in the earlier sections of this document. These formulations contain both hydrophobic and hydrophilic components that are combined in a unique, unanticipated manner (in prior art) combinations in a concentrated form that are self-emulsified as revealed in the examples requiring little or no mechanical energy for the emulsification step. Post emulsification processing can include: 1) dilution with additional sterile water or other additives such as buffering agents (to control concentration and pH to meet physiological compatibility and comfort needs); 2) added steps to reduce and optimize micelle size to the sub-micron or nano-scale that can include high frequency ultrasonic agitation; 3) packaging and sterilization via a variety of methods (including aseptic filtration, sterilizing heat, use of ionizing radiation) for storage stability and safety for use by patients and administering physicians. The examples are not intended to limit in any way the formulations that can potentially be generated using alternative components within the various classes of compounds described in this document and within the scope of the disclosures resulting in self-emulsifying formulations for use in topically applied applications, including but not limited to use as an eye drop in the practice of ophthalmology, both as currently practiced but as may be envisioned, identified and needed in the future.

### Inventive Compositions and Processes for Their Preparation:

The inventive formulations and the self-emulsification processes for producing them will be described in principle in detail in this section. The inventive process for preparing the emulsified formulations of this invention require emulsifying agents, e.g. surfactants, having specific properties suited to the process of self emulsification. These can include ionic or non-ionic surfactants. However, non-ionic surfactants are generally preferred as they are compatible for use with a wide variety of emulsifiable components whereas ionic surfactants generally have more limited utility for components that are both ionic and non-ionic in their chemical nature. There are two preferred classes of non-ionic surfactants: 1) polysorbates including polysorbate 80 and the 2) Polyoxamers-block copolymers of polypropylene oxide and ethylene oxide that also are thermally reversible in that they become viscous as temperature rises near normal body temperatures. Thickeners, to increase viscosity and increase retention time after application to the eye surface are also preferred and include hyaluronic acid or its sodium salts, xanthan (a polysaccharide) and certain Pluronics that demonstrate inverse temperature-vs-viscosity characteristics (viscosity of emulsions with it increase near body temperature rather than decrease) at specific concentrations. Preferred Pluronics include F127, F108, F105. Pluronic F84 was used in the examples as it operates as both a thickener and as an emulsifying agent for Part B.

These options are useful for various applications including topical use. The hydrophilic-lipophilic balance of a surfactant is a measure of the degree for which it is hydrophilic or lipophilic, determined by calculating values for the different regions of the molecule, as described by Griffin in 1949 (Dartt, Darlene A., Hodges, Robin R., Tear Film Mucins: Front Line Defenders of the Ocular Surface: Comparison with Airway and GI Tract Mucins, exp eye res. 2014 Cec: 117-62-78; doi: 10.1016/i.exer.2013.07-027). The role of high molecular weight hyaluronic acid in mucoadhesion on an ocular surface model, Galesso et al., Journal of the Mechanical Behavior of Biomedical Materials 143 (2023) 105908, https://doi.org/10.1016/j.jmbbm.2023.105908].

Ascorbic acid esters, including ascorbic acid palmitate. L-ascorbic acid alkyl esters (ASCn) are molecules of pharmaceutical interest for their amphiphilic nature and proposed antioxidant power. In contrast to L-ascorbic acid, ASC (n) with different acyl chain lengths behaved stably upon oxidation and a tautomeric isomerization was observed. In Langmuir films, when the ascorbic ring has a negative charge, ASC14 and ASC16 form stable monolayers, contrary to ASC10 and ASC12. ASC16 films showed transition from liquid-expanded (LE) to liquid-condensed phase, whereas ASC14 showed only an LE phase. When ASCn are mainly neutral, ASC14 showed phase transition from LE to a crystalline phase, as previously reported for ASC16. The two-dimensional domains displayed crystal-like shapes with anisotropic optical activity when interacting with the polarized light under Brewster angle microscopy. The compounds with the longer acyl chain (ASC16, ASC14 and ASC12) exhibited good surface activity, forming Gibbs monolayers. They also were able to penetrate into phospholipid monolayers up to a critical point of 45-50 mN/m. The I-palmitoyl-2-oleoylphosphatidylcholine/ASCn films showed LC and/or crystalline domains only for ASC16. This study provides valuable evidence regarding the stability and surface properties of this drug family, and casts light into the differential interaction of these drugs with lipid membranes, which is important for understanding its differential pharmacological activity.

In addition to surfactants, hydrophilic, polymeric components can be added to raise the viscosity of the formulations to increase retention time in the eye resulting in increased drug delivery to the target tissues. These components produce hydrogels that swell and retain water and interact with the aqueous tears and the tear film naturally present in the eye and the mucin layer, the inner most layer of the anterior segment, Topically applied therapeutics are formulated to be assimilated into the tears quickly and comfortably and thereby deliver the active compounds to the surface of the eye. Macular degeneration and diabetic retinopathy are diseases of the posterior segment (retina included) of the eye, while the eye drops are placed on the anterior portion of the eye, requiring transport through tissues to reach the posterior region to be effective in treatment. The preferred hydrophilic polymeric components of this invention are hyaluronc acid (or its sodium salts), polyoxomers (Pluronics) EO-PO-EO triblock copolymers, or specific polysaccharides including xanthan—a polysaccharide produced by bacteria on surfaces of green plants and produced synthetically—that are safe and used in FDA approved eye drop products.

Hyaluronic acid (HA) improves the stability of the tear film by hydration and lubrication. Mucoadhesion is related to the ocular residence time and therefore to the effectiveness of the eye drops. The ocular residence time of the HA formulation is correlated with the ability of HA to create specific strong interactions in the ocular surface with the mucus layer, mainly composed of a mixture of secreted mucins (MUC; gel forming MUC5AC and MUC2) and shed membrane-bound soluble mucins (MUC1, MUC4, and MUC16). Dry eye disease (DED) is a multifactorial pathology of the preocular tear film with possible damage to the ocular surface classified in two types: (1) aqueous-deficient dry eye and (2) evaporative dry eye, caused by a decrease in goblet cell density that reduces MUC expression and/or by meibomian gland dysfunction, that results in a drop in the lipidic fraction of the tear film.

Adding these components to the formulations also increases the viscosity of the resulting eye drop making it easier to dispense into the eye with control.

Pharmaceutical excipients are included as potential ingredients for the compositions described in this document. The phrase “functional excipient” usually implies that the excipient has specific functionality such as—carboxyl,-hydroxyl,-amino,-thiol, etc. For purposes of this filing, it may also mean that the excipient may ave additional functional characteristics in that the compound participates as an active component of a biochemical pathway as a solvent, co-factor, transporter, receptor agonist or antagonist, an enzyme, as a catalyst,an electron transfer reducing or oxidizing agent, as a eutectic agent, an anti-microbial or anti-viral agent, a photosensitizer, a UV absorber, or other class of compounds having specific features important to the process of manufacture and/or to the targeted therapeutic characteristics of the formulation. Functional excipients can be natural products, obtained from plants, or other living species (fungi, insects, algae including microalgae) that could include, for example, enzymes such as protease or kinase enzymes. All publications, patents, patent applications and articles cited are incorporated herein by reference in their entireties.

### Further Examples

The materials employed in the examples are all commercially, intended for human use, and available at least from vendors cited in Table 1, unless otherwise indicated. Self-emulsifying formulations of this invention are directed for use as topically applied medicaments, including those uses in the practice of ophthalmology in treating eye disease including age-related macular degeneration, diabetic retinopathy, and myopia and others such as glaucoma, dry eye disease. They are produced from components that are generally water soluble (hydrophilic) along with others that are water insoluble (hydrophobic). Combining these two-water soluble and water insoluble-requires compositions and methodologies that produce emulsions of the oil in water type and processing that facilitates that result. In some cases, it has proven possible to produce solutions containing water insoluble components in water only by the order or addition of the components to the solution without making an emulsion. This will be explained and demonstrated by example.

Part A—the aqueous component containing water soluble or water dispersible compounds or agents, and Part B-lipophilic components that are not water soluble or readily dispersible or miscible. The intimate blending of these two main components-Part A and Part B—is essential to the ultimate functionality of the end-product—for example, an eye drop or topical cream or lotion that can be utilized as a key part of an effective treatment for specific medical conditions. The most effective delivery system for such an intimate blend is an emulsion having very small micelle structures less than about 100 nanometers in dimension that is stable long term for weeks or months for use by physicians and patients. Means and compositions for producing this class of emulsions are the subject of this invention. Specifically, well known biological agents that are effective in treating the outlined medical conditions can be incorporated into these emulsions for effective delivery. Topical delivery to the diseased tissues represents a targeting strategy for treatment as opposed to systemic delivery via oral means that requires passage through the gut and then to the bloodstream and finally to the diseased tissues in order to effect a therapeutic result. While this process can be effective, it is generally not efficient as the passage through the gut and other tissues before arrival at the target tissue results in what is referred to as the 1st pass effect—a loss of the concentration and chemical composition of the active ingredients due to adverse metabolic reactions for example hydrolysis or enzymatic breakdown of chemical structures. Hence topical delivery can avoid these complications if the therapeutic components can be applied directly to the diseased tissues or be to them by transported to them by localized diffusion or transmembrane transport.

What is claimed:

1. An aqueous liquid composition for use in treating genetically resultant conditions in the eye comprising levo-dopamine and its derivatives and an antioxidant combination of glutathione and cobalamin.

2. The composition of claim 1 wherein the levo-dopamine is selected from the group consisting of levodopa, carbidopa, levodopa metabolite, and 3-O-methyldopa.

3. The composition of claim 1 wherein the levo-dopamine is a combination of levodopa and at least one other levo-dopamine selected from the group consisting of carbidopa, levodopa metabolite, and 3-O-methyldopa.

4. The composition of claim 1 further comprising ascorbic acid or an ascorbic acid ester.

5. The composition of claim 1 wherein the levodopa is a combination of levodopa and carbidopa.

6. The composition of claim 1 further comprising an inert viscosity increasing polymer.

7. The composition of claim 1 further comprising dissolved Vitamin E.

8. The composition of claim 6 further comprising dissolved Vitamin E.

9. A method of at least reducing genetic disease effects in the eyes of mammals comprising depositing a liquid composition onto an exposed surface of the eye, the liquid composition comprising a levo-dopamine and an antioxidant combination of glutathione and cobalamin.

10. The method of claim 9 wherein the liquid composition is applied as drops onto an exposed surface of the eye of a mammal.

11. The method of claim 10 wherein the levo-dopamine is selected from the group consisting of levodopa, carbidopa, levodopa metabolite, and 3-O-methyldopa.

12. The method of claim 10 wherein the levo-dopamine is a combination of levodopa and at least one other levo-dopamine selected from the group consisting of carbidopa, levodopa metabolite, and 3-O-methyldopa.

13. The method of claim 10 wherein the liquid composition further comprises ascorbic acid or an ascorbic acid ester.

14. The method of claim 10 wherein the levo-dopamine is a combination of levodopa and carbidopa.

15. The method of claim 10 wherein the liquid composition further comprises an inert viscosity increasing polymer.

16. The method of claim 10 wherein the liquid composition further comprises dissolved Vitamin E.

17. The method of claim 14 wherein the liquid composition further comprises dissolved Vitamin E.

18. The liquid composition of claim 1 provided in a bottle with an eye dropper comprising ingredients selected from the group consisting of a) at least one of L-DOPA, prodrug including Methyldopa, Etilevodopa (ethyl ester of Levodopa), or Carbidopa, b) glutathione and c) Vitamin B12 antioxidants

19. The liquid composition of claim 1 further comprising melatonin and Vitamin C.

20. The liquid composition of claim 1 further comprising melatonin, Vitamin C, and a polyoxamer dispersing agent.

21. The liquid composition of claim 1 provided in a bottle with an eye dropper comprising ingredients selected from the group consisting of a) L-DOPA, Methyldopa, Etilevodopa, Carbidopa, b) glutathione, c) Vitamin B12, d) Vitamin E, e) Tween 80 or Polyoxamer 84.

22. The liquid composition of claim 4 further comprising hyaluronic acid.

23. Method for treating eye diseases including macular degeneration, diabetic retinopathy, or myopia with the liquid composition of claim 1 by applying the liquid composition onto an eye of a human being.
